(19) United States
(12) Patent Application Publication
(10) Pub. No.: US 2019/0175110 A1
(43) Pub. Date: Jun. 13, 2019

(54) AUTOEXPANDABLE DEVICES FOR OBTAINING IMPEDANCE, PRESSURE, AND OTHER MEASUREMENTS IN THE GASTROINTESTINAL TRACT AND OTHER ORGANS

(71) Applicants: Hans Gregersen, Ma On Shan, New Territories (HK); Ghassan S. Kassab, La Jolla, CA (US)

(72) Inventors: Hans Gregersen, Ma On Shan, New Territories (HK); Ghassan S. Kassab, La Jolla, CA (US)

(21) Appl. No.: 16/322,381

(22) PCT Filed: Jul. 31, 2017
(86) PCT No.: PCT/US17/44707

§ 371 Date: Jan. 31, 2019

Related U.S. Application Data
(60) Provisional application No. 62/369,192, filed on Jul. 31, 2016.

(51) Int. Cl. A61B 5/00; A61B 5/03; A61B 5/053; A61B 5/07; A61B 1/04; A61L 31/00; A61L 31/14; A61B 5/145; A61B 5/1473; A61B 5/0488; A61N 2/02; A61N 1/36

(52) U.S. Cl. CPC A61B 5/6861; A61B 5/036; A61B 5/0538; A61B 5/073; A61B 1/041; A61L 31/005; A61B 2090/3966; A61B 5/14539; A61B 5/1473; A61B 5/0488; A61N 2/02; A61N 1/36007; A61B 2562/06; A61L 31/14

(57) ABSTRACT

Autoexpandable devices for obtaining impedance, pressure, and other measurements in the gastrointestinal tract and other organs. In an exemplary embodiment of a capsule of the present disclosure, the capsule comprises an outer shell configured to dissolve or otherwise be digested within a stomach after ingestion, an expandable material positioned within the outer shell, the expandable material configured for expansion within the stomach after the outer shell has dissolved, and at least one element or sensor selected from the group consisting of an impedance element and a pressure sensor.

### PRIORITY

The present disclosure is related to, and claims the priority benefit of, U.S. Provisional Patent Application Ser. No. 62/369,192, filed Jul. 31, 2016, the contents of which are incorporated into the present disclosure directly and by reference in their entirety.

### BACKGROUND

There is considerable interest regarding the development of medical devices that can record data from inside the body in a wireless fashion without the requirement of extensive medical procedures like endoscopy. There is also significant interest in integrating such data with various information technology (IT) platforms to make the data available for individuals for health purposes, such as in connection with adjustments of their life quality and performance.

Recent advances in technology relate to devices configured for expansion within the stomach for obesity treatment. Obesity has reached epidemic proportions worldwide, including the United States and China for example, and represents a large number of physician and hospital visits each year. Obesity is a major cause of diabetes and cardiovascular disease causing reduced life quality and death. Therefore, there is a huge interest in development of new obesity treatments. Even for persons who are just slightly overweight, there is a need for development of new ways to reduce weight, especially for young people believing that they weigh just a few pounds or kilograms too much. Said capsules, when swallowed, will make the users feel less hungry and therefore they will eat less. These types of capsules can be readily swallowed and are intended to remain in the stomach for hours or days after the expansion. However, said capsules are not currently able to obtain any sort of data regarding the user's gastrointestinal system or from other organs in the body of animals or humans

In view of the foregoing, capsules configured to be swallowed, to result in the user eating less, and also to obtain meaningful gastrointestinal data so to assist with potential obesity treatment, for example, would be well received in the marketplace.

### BRIEF SUMMARY

The present disclosure includes disclosure of capsules, consoles, systems, and methods of using the same. A capsule, as referenced herein, is a swallowable item having an outer portion that dissolves in the stomach, which has it dissolves reveals an expandable material and other items as referenced herein.

In an exemplary embodiment of a capsule of the present disclosure, the capsule comprises an impedance element; and/or a pressure sensor; and/or one or more additional elements; wherein the capsule is surrounded by an outer shell configured to dissolve or otherwise be digested in the stomach; and wherein the capsule contains an expandable material configured to expand in the stomach. The capsule is the other layer made of gelatin or similar that quickly will dissolve in the stomach. It contains the expandable material with the embedded electronics. Since the expandable material may be particles or consist of rather small units, then a membrane (or kind of net or mesh) can be used, in certain embodiments, to hold it together (otherwise it will pass from the stomach into the intestine). Different materials for the membrane and the expandable membrane, so to dissolve at different time points (for example after few hours or after weeks inside the stomach).

In an exemplary embodiment of a capsule of the present disclosure, the impedance element comprises two detection electrodes positioned in between two excitation electrodes.

In an exemplary embodiment of a capsule of the present disclosure, the impedance element is configured to obtain impedance data within a gastrointestinal tract.

In an exemplary embodiment of a capsule of the present disclosure, the pressure sensor is configured to obtain pressure data within a gastrointestinal tract from one or more pressure sensors.

In an exemplary embodiment of a capsule of the present disclosure, the one or more additional elements are selected from the group consisting of a camera configured to obtain visual data within a gastrointestinal tract, a pH sensor configured to obtain pH data within a gastrointestinal tract, a radiopaque marker configured to be detected using a scanner configured to detect a radiopaque marker within the body, an electromagnetic marker configured to be detected using a scanner configured to detect an electromagnetic marker within the body, a mucosa-attached electromagnetic stimulator (through non-invasive external stimulation), and/or a strain gauge having chemosensors embedded in the biological material. This is not an exhaustive listing of additional elements that could be used.

In an exemplary embodiment of a capsule of the present disclosure, the expandable material contains electrochemical means for active movement inside the stomach such as robotic arms or propellers that can aid movement of the device or even temporarily attach it to the stomach wall.

In an exemplary embodiment of a capsule of the present disclosure, the expandable material comprises a fungus (dried or partially dried)

In an exemplary embodiment of a capsule of the present disclosure, the expandable material is surrounded by a mesh configured to expand or unfold as expandable material expands.

In an exemplary embodiment of a capsule of the present disclosure, the capsule is configured to communicate with a console.

In an exemplary embodiment of a capsule of the present disclosure, the capsule further comprises one or more of the following: a computer element; and/or a wireless transmitter; and/or a wireless receiver; and/or a power source.

In an exemplary embodiment of a capsule of the present disclosure, the capsule comprises an outer shell configured to dissolve or otherwise be digested within a stomach after ingestion; an expandable material positioned within the outer shell, the expandable material configured for expansion within the stomach after the outer shell has dissolved; and at least one element or sensor selected from the group consisting of an impedance element and a pressure sensor.

In an exemplary embodiment of a capsule of the present disclosure, the at least one element comprises the impedance element.

In an exemplary embodiment of a capsule of the present disclosure, the impedance element comprises two detection electrodes positioned in between two excitation electrodes.

In an exemplary embodiment of a capsule of the present disclosure, the impedance element is configured to obtain impedance data within a gastrointestinal tract.

In an exemplary embodiment of a capsule of the present disclosure, the at least one element comprises the pressure sensor.

In an exemplary embodiment of a capsule of the present disclosure, the pressure sensor is configured to obtain pressure data within a gastrointestinal tract.

In an exemplary embodiment of a capsule of the present disclosure, the at least one element comprises the impedance element and the pressure sensor.

In an exemplary embodiment of a capsule of the present disclosure, the impedance element comprises two detection electrodes positioned in between two excitation electrodes, wherein the impedance element is configured to obtain impedance data within a gastrointestinal tract, and wherein the pressure sensor is configured to obtain pressure data within a gastrointestinal tract.

In an exemplary embodiment of a capsule of the present disclosure, the at least one element or sensor is/are coupled to the expandable material.

In an exemplary embodiment of a capsule of the present disclosure, the capsule further comprises a camera configured to obtain visual data within a gastrointestinal tract.

In an exemplary embodiment of a capsule of the present disclosure, the capsule further comprises a pH sensor configured to obtain pH data within a gastrointestinal tract.

In an exemplary embodiment of a capsule of the present disclosure, the capsule further comprises a radiopaque marker configured to be detected using a scanner configured to detect the radiopaque marker within a mammalian body.

In an exemplary embodiment of a capsule of the present disclosure, the capsule further comprises an electromagnetic marker configured to be detected using a scanner configured to detect the electromagnetic marker within a mammalian body.

In an exemplary embodiment of a capsule of the present disclosure, the capsule further comprises a mucosa-attached electromagnetic stimulator.

In an exemplary embodiment of a capsule of the present disclosure, the capsule further comprises a strain gauge having chemosensors.

In an exemplary embodiment of a capsule of the present disclosure, the expandable material comprises a fungus.

In an exemplary embodiment of a capsule of the present disclosure, the fungus comprises a black fungus

In an exemplary embodiment of a capsule of the present disclosure, the expandable material is surrounded by a mesh configured to expand as expandable material expands.

In an exemplary embodiment of a capsule of the present disclosure, the at least one element or sensor is/are coupled to the mesh.

In an exemplary embodiment of a capsule of the present disclosure, the at least one element or sensor is/are contained within the mesh.

In an exemplary embodiment of a capsule of the present disclosure, the capsule further comprises a pH sensor configured to obtain pH data within a gastrointestinal tract.

In an exemplary embodiment of a capsule of the present disclosure, he at least one element comprises the impedance element and the pressure sensor, the impedance element comprises two detection electrodes positioned in between two excitation electrodes, the impedance element is configured to obtain impedance data within a gastrointestinal tract, the pressure sensor is configured to obtain pressure data within a gastrointestinal tract, the expandable material comprises a fungus, and the expandable material is surrounded by a mesh configured to expand as expandable material expands.

In an exemplary embodiment of a capsule of the present disclosure, the capsule is configured to communicate with a console.

In an exemplary embodiment of a capsule of the present disclosure, the capsule further comprises a wireless transmitter and a wireless receiver.

In an exemplary embodiment of a capsule of the present disclosure, the wireless transmitter is configured to transmit data obtained by at least one of the at least one element or sensor to a console, and wherein the wireless receiver is configured to receive console data from the console.

In an exemplary embodiment of a capsule of the present disclosure, the capsule further comprises a substrate, wherein at least one of the at least one element or sensor is coupled to the substrate.

In an exemplary embodiment of a capsule of the present disclosure, the capsule further comprises a printed circuit configured to measure electrical/electromyography (EMG) activity within a colon.

In an exemplary embodiment of a capsule of the present disclosure, the at least one element comprises the impedance element and the pressure sensor wherein the impedance element comprises two detection electrodes positioned in between two excitation electrodes, wherein the impedance element is configured to obtain impedance data within a gastrointestinal tract, and wherein the pressure sensor is configured to obtain pressure data within a gastrointestinal tract.

In an exemplary embodiment of a capsule of the present disclosure, the capsule further comprises a gyroscope configured to obtain gyroscopic data within a digestive tract.

In an exemplary embodiment of a capsule of the present disclosure, the capsule is configured to deliver electrical current to stimulate motility of a colon to induce defecation.

In an exemplary embodiment of a capsule of the present disclosure, the at least one element comprises the impedance element, and wherein the impedance element is configured to deliver the electrical current.

In an exemplary embodiment of a capsule of the present disclosure, the capsule further comprises an electrical stimulating element configured to deliver the electrical current.

In an exemplary embodiment of a capsule of the present disclosure, the capsule further comprises a computer element.

In an exemplary embodiment of a capsule of the present disclosure, the capsule further comprises a movement element coupled thereto and/or embedded therein, the movement element configured to facilitate movement of the capsule within the stomach.

In an exemplary embodiment of a capsule of the present disclosure, the capsule further comprises an attachment element coupled thereto and/or embedded therein, the attachment element configured to facilitate engagement and ultimate disengagement of the capsule to a portion of a gastrointestinal tract.

In an exemplary embodiment of a capsule of the present disclosure, the capsule comprises an outer shell configured to dissolve or otherwise be digested within a stomach after ingestion; an expandable material positioned within the outer shell, the expandable material configured for expansion within the stomach after the outer shell has dissolved; a mesh surrounding the expandable material and configured to expand as expandable material expands; an impedance element configured to obtain impedance data within a gastrointestinal tract; a pressure sensor configured to obtain pressure data within the gastrointestinal tract; wherein the impedance element and the pressure sensor are coupled to a substrate; and wherein the substrate is surrounded by the mesh or coupled to the mesh.

In an exemplary embodiment of a capsule of the present disclosure, the expandable material comprises a fungus.

In an exemplary embodiment of a capsule of the present disclosure, the capsule further comprises a wireless transmitter and a wireless receiver, wherein the wireless transmitter is configured to transmit data obtained by at least one of the at least one element or sensor to a console, and wherein the wireless receiver is configured to receive console data from the console.

In an exemplary embodiment of a capsule of the present disclosure, the capsule is configured to deliver electrical current to stimulate motility of a colon to induce defecation.

In an exemplary embodiment of a method of obtaining data of the present disclosure, the method comprises the step of operating an exemplary capsule of the present disclosure within a digestive tract.

In an exemplary embodiment of a method of obtaining data of the present disclosure, the step of operating is performed to obtain impedance data from the impedance element and pressure data from the pressure sensor.

In an exemplary embodiment of a method of obtaining data of the present disclosure, the capsule further comprises a wireless transmitter configured to transmit the impedance data and the pressure data to a console.

In an exemplary embodiment of a method of obtaining data of the present disclosure, the method further comprises the step of electrically stimulating a colon of the digestive tract using the capsule to induce defecation.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a capsule, according to an exemplary embodiment of the present disclosure;

FIG. 2 shows a block component diagram of a console and a system, according to an exemplary embodiment of the present disclosure;

FIG. 3 shows components of a computer element, according to an exemplary embodiment of the present disclosure; and

FIG. 4 shows a capsule and a console communicating with one another over a network, according to an exemplary embodiment of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes disclosure of capsules, configured to be swallowed, to result in the user eating less, and also to obtain meaningful gastrointestinal data so to assist with potential obesity treatment, for example. Methods of using the same to treat a gastrointestinal and/or other weight-related condition are disclosed herein as well. The device may assist with measurement of important physiological variables from other organs such as the cardiovascular system, respiratory function, diagfragmatic movements as well as body homeostasis exemplified with temperature measurements. Important respiratory and cardiovascular data may be obtained by microphones or other technologies that measure vibration. It will also give information about abdominal pressure which will be an indicator of the subject's posture and intraabdominal load. Pattern recognition will help to distinguish different patterns, for example of sleep/awake cycles, eating patterns, gastric motility patterns, abnormal contractility, intestino-gastric reflux of bile acid, and excessive respiration such as hyperventilation.

In various embodiments, said capsules also serve as a carrier for electromechanical sensors and/or as a drug delivery system. Regardless of whether or not capsules of the present disclosure are used for weight loss purposes, as a drug delivery system (GLP (Glucagon-like peptide)-satiety hormone or Ghrelin-hunger hormone inhibitors), a nutrient-delivering system, and/or for carrying an electromechanical device that will measure physiological parameters (e.g., pressure or dimensional changes for motility), it is important to control the duration the device is located in the stomach. Such durations can be tightly controlled by using different materials for the expanding material and for the embracing membrane. Alternatively, material that can be dispersed or broken down by, for example, external ultrasound can also be used. The control of ingestion of amount and number of capsules as well as their breakdown duration allows for control of the expanded volume at given times. In this way the limitations of the fixed-volume stomach balloons for weight-loss can be avoided.

The following describes a process that will shed light on different variations of the products, the possible routes to the market and prototypes for several phases. It is separated into phases that to some extent are overlapping.

In at least one embodiment of a capsule of the present disclosure, the capsule comprises a material configured for expansion within the gastrointestinal tract and comprising at least one of an element to obtain impedance data within the gastrointestinal tract and/or an element configured to obtain pressure data within the gastrointestinal tract. Said elements can be connected to various consoles or platforms, such as the Wechat platform, Whatsapp or health platforms, for example, so that data from said elements can be obtained and processed, for example, by said platform. This will provide an opportunity for biofeedback and will furthermore allow collection of large data sets providing information about the health of the general population.

An exemplary capsule of the present disclosure can comprise gelatin, and be at least partially surrounded or covered by a wrap (which may also be referred to as a coating, mesh, or membrane). Such an expandable material (biological, such as dried black fungus, or artificial) could, in various embodiments, expand up to 15 times or more from their original size when exposed to liquid, and be held together by a wrap as referenced above. Depending on the material used, the time the capsule is located in the stomach can be controlled. Said materials would be used in lieu of existing stomach products, such as balloons, fibramin (cognac root), or other stomach fillers. Said capsules, as referenced herein, can relate to the general purpose of weight loss for overweight patients, or “shaping.”

Various capsules of the present disclosure can further comprise a nutritive element (food) and/or a drug desired to be introduced into the stomach. For some purposes it may be an advantage to release caloric nutritive elements since it will delay gastric emptying that is facilitated by powerful contractions in the antrum of the stomach and by pyloric sphincter relaxations. Hence, it may be less likely that the expanded device will pass into the intestine which at least in some situations will be judged as an unwanted event.

Various capsules of the present disclosure, in addition to having elements to obtain impedance data and/or pressure data within the gastrointestinal tract, can also comprise one or more of a camera, a gyroscope, a pH sensor, a radiopaque marker, an electromagnetic marker, a mucosa-attached electromagnetic stimulator (through non-invasive external stimulation), a strain gauge and chemosensors embedded in the biological material, and the like. Said elements and/or sensors can comprise various photonic sensors, carbon nanotube sensors, and infrared sensors, which may include body-powered sensing (ETH) technologies. Said elements can then be configured to transmit data obtained in the gastrointestinal tract wirelessly to a console configured to receive and process said data. Wireless transmission of data in real time for improved efficiency and user-satisfaction including symptom score evaluation and possible disease detection could be had in various embodiments referenced herein. Alternatively the data can be stored on a chip and transmitted after the device leaves the body.

Exemplary capsules of the present disclosure, depending on embodiment, are configured to a) measure stomach function in terms of one or more of pressure profiles, acidity, gastric emptying and duodeno-gastric reflux b) measure other body functions such as respiratory function over an extended time period, digestive-sleep interactions, abdominal blood pressure, and the like, and c) tightly control the amount of drug delivery to treat stomach problems or whole body problems.

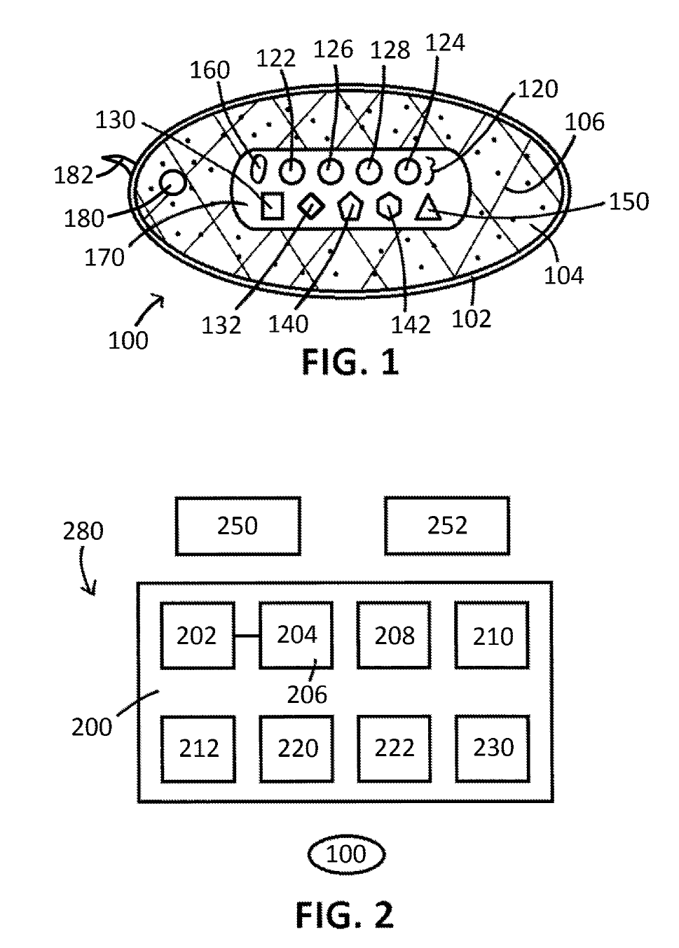

FIG. 1 shows an exemplary capsule 100 of the present disclosure. As shown therein, capsule 100 comprises an outer shell 102 covering said capsule 100, whereby outer shell 102 is configured to dissolve within the stomach after being ingested. An expandable material 104 is positioned within outer shell 102, so that when outer shell 102 is dissolved and liquid from within the stomach comes into contact with expandable material 104, expandable material 104 can expand after contacting said liquid. Expandable material 104 can be at least partially surrounded by a mesh 106, such as shown in FIG. 1, so that when expandable material 104 expands within the stomach, mesh 106 also expands so to retain the expandable material 104 within mesh 106. In some embodiments, expandable material 104 may comprise various pieces of expandable material 104 that are not connected to one another, and in such an embodiment, for example, mesh 106 can retain each piece of expandable material 104 within said mesh 106.

Outer shell 102 can dissolve relatively quickly after being swallowed, such as in a matter of seconds to a few minutes, ranging from 1 second to 5 minutes or longer. Expandable material 104 can expand up to, for example, two, three, four, five, ten, twenty, fifty, one hundred, or less or more, times its original size/volume, after being exposed within the stomach (after outer shell 102 has partially or fully dissolved or been partially or fully digested, for example). Expandable material 104 could expand, for example, and have an expansion length (such as from one relative side of expandable material 104 to an opposite relative side of expandable material 104) of 3 cm, 4 cm, 5 cm, 7 cm, 10 cm, or more or less. In at least one embodiment, expandable material 104 comprises a fungus, such as black fungus, that would be dry or relatively dry within capsule 100 at the time capsule 100 is swallowed. Expandable material 104, after expansion, would occupy space within the stomach that is more space than capsule 100 occupies right after being swallowed, and the larger space would help make the patient feel like more food is in his or her stomach, providing a feeling of satiety even though less food is ingested, for example. The patient may swallow several capsules 100 at different time points to avoid that the stomach always will have the same amount of expandable material 104 inside. Mesh 106 would then be configured to retain the expanded expandable material, and various other elements of capsule 100, such as a substrate 170 and elements thereon or connected thereto, as referenced herein.

Capsules 100 of the present disclosure may also be referred to and/or configured as pills, pellets, and the like, and be sized and shaped so to be readily swallowed by a patient.

In an exemplary embodiment of a capsule 100 of the present disclosure, the expandable material 104, or other components of capsule 100, contains electrochemical means for active movement inside the stomach, such as robotic arms or propellers, which can aid movement of the device or even temporarily attach it to the stomach wall. For example, and as shown in FIG. 1, an exemplary capsule 100 of the present disclosure can comprise one or more movement elements 180 coupled thereto and/or embedded therein, and/or can comprise one or more attachment elements 182 coupled thereto and/or embedded therein, whereby movement elements 180, such as robotic arms, propellers, or other mechanical devices configured to facilitate movement of a device, are configured to facilitate movement of the capsule 100 within the stomach, and wherein attachment elements 182, configured as barbs, hooks, or the like, are configured to facilitate engagement and ultimate disengagement of capsule 100, as may be desired, to locations within the gastrointestinal tract.

Exemplary capsules 100 of the present disclosure are also configured so to obtain impedance data and/or pressure data within a gastrointestinal tract. To accomplish the same, exemplary capsules 100 of the present disclosure may comprise various electrical components, sensors, and the like, as described in further detail herein. For example, and exemplary capsule 100 of the present disclosure may comprise an impedance element 120, as shown in FIG. 1, configured to obtain impedance data within the gastrointestinal tract. Such an impedance element 120, for example, may comprise several electrodes, such as a first excitation electrode 122 and a second excitation electrode 124 configured to generate an electric field that is detectable using a first detection electrode 126 and a second detection electrode 128, for example. Detection electrodes 126, 128 could be physically positioned in between excitation electrodes 122, 124 for example, so to obtain impedance data as desired.

Exemplary capsules 100 of the present disclosure may contain various elements in lieu of or in addition to impedance element 120 noted above, such as, for example, a pressure sensor 130 configured to obtain pressure data within the gastrointestinal tract, and optionally one or more additional elements 132 which may comprise, for example, a camera with a light source configured to obtain visual data within a gastrointestinal tract, a pH sensor configured to obtain pH data within a gastrointestinal tract, a radiopaque marker configured to be detected using a scanner configured to detect a radiopaque marker within the body, an electromagnetic marker configured to be detected using a scanner configured to detect an electromagnetic marker within the body, a mucosa-attached electromagnetic stimulator (through non-invasive external stimulation), a gyroscopic sensor (a gyroscope) configured to obtain gyroscopic data, including but not limited to gyroscope-based angle data with the digestive tract, and/or a strain gauge and chemosensors embedded in the biological material. Said data obtained using impedance element 120, pressure sensor 130, and/or one or more additional elements 132 can be transmitted from capsule 100 from within the body, for example, to a console 200 outside of the body, by way of a wireless transmitter 140 within said capsule 100. Capsule 100 may also receive instructions from console 200, such as instructions to operate or cease operation of one or more of impedance element 120, pressure sensor 130, and/or one or more additional elements 132, using a wireless receiver 142 within said capsule 100. In various embodiments, wireless transmitter 140 could also receive instructions/data, so said transmitter 140 could be considered as a transmitter/receiver.

In at least one embodiment, one or more additional elements 132 may comprise a printed circuit, such as a printed circuit board (PCB), which may be/comprise a substrate 170 as referenced in further detail herein, configured to measure electrical/electromyography (EMG) activity (exemplary data) within the colon, for example. Said data can, in various embodiments, be obtained in addition to various mechanical measurements, such as pressure data obtained by pressure sensor 130 as referenced herein, cross-sectional area (CSA) data determined by impedance data obtained using impedance element(s) 120, and potential gyroscope-based angles obtained using one or more additional elements 132.

In at least one embodiment of a capsule 100 of the present disclosure, said capsule 100 is configured to deliver electrical current to stimulate motility of the colon, such as to, for example, induce defecation for individuals with constipation, and the like. In such an embodiment, impedance elements 120 (such as electrodes 122, 124), or one or more additional elements 132 configured as an electrical stimulating element, can deliver an electric current, powered by power source 160, to stimulate motililty of the colon.

A computer element 150, as referenced in further detail herein, could also be positioned within capsule 100, and connected (via wires, traces, and/or wirelessly) to one or more of impedance element 120 (including, for example, electrodes 122, 124, 126, and/or 128), pressure sensor 130, one or more additional elements 132, wireless transmitter 140, and/or wireless receiver 142, so to, for example, control operation of said items. Computer element 150 could direct operation of said items without input from console 200, and/or could direct operation of said items with input from console 200, such as instructions from console 200 received by wireless receiver 142 and transmitted to computer element 150.

Impedance element 120 (including, for example, electrodes 122, 124, 126, and/or 128), pressure sensor 130, one or more additional elements 132, wireless transmitter 140, wireless receiver 142, and/or computer element 150 could be powered using a power source 160, such as a battery. Power source 160 could be a single-use battery or a rechargeable battery, as may be desired. Alternative the energy supply can come from body-powered sources and elements.

Impedance element 120 (including, for example, electrodes 122, 124, 126, and/or 128), pressure sensor 130, one or more additional elements 132, wireless transmitter 140, wireless receiver 142, computer element 150, and/or power source 160 could be connected to a substrate 170, such as shown in FIG. 1. Such a substrate 170 could comprise plastic and/or metal, for example, and be configured to remain within capsule 100 after outer shell 102 has dissolved and after expandable material 104 has been expanded.

Capsules 100 of the present disclosure are configured to obtain data/information with the gastrointestinal tract, including the stomach, the small intestine, the large intestine, various junctions and/or sphincters therein, and the anus, including, but not limited to, impedance data, pressure data, pH data, and other data as referenced herein. Capsules 100 can be safely swallowed and excreted.

Impedance data can be used to determine various measurements, such as cross-sectional areas, diameters, etc., within luminal organs, as referenced within U.S. Pat. No. 7,454,244 to Kassab et al., the contents of which are incorporated herein in their entirety by reference.

Various systems can also be established in connection with various capsules 100 of the present disclosure. FIG. 2 shows components of an exemplary console 200 of the present disclosure. As shown therein, console 200 comprises a processor 202 (such as a microprocessor) connected to or otherwise in communication with a storage medium 204. Software 206 (computer instructions) can be stored within storage medium 204 and accessed by processor 202 to operate console 200 as described herein. Exemplary consoles 200 of the present disclosure may further comprise a display 208 (such as a computer screen, monitor, LCD, etc.) configured to display information therein, and may be configured to communicate with an input device 250 (such as a mouse, keyboard, touchpad, touchscreen, stylus, microphone, smartphone, tablet, etc.), either wirelessly or via wired connection to console 200, such as via input port 210. Exemplary consoles 200 of the present disclosure may also be configured to communicate with an output device 252 (such as a monitor, screen, speaker, printer, etc.), either wirelessly or via wired connection to console 200, such as via input port 212.

Exemplary consoles 200 of the present disclosure may further comprise a wireless transmitter 220 configured to transmit data/information to capsule 100, and may also comprise a wireless receiver 222 configured to receive data/information from capsule 100. Said data/information could include instructions for operation of various items of capsule 100, such as impedance element 120 (including, for example, electrodes 122, 124, 126, and/or 128), pressure sensor 130, one or more additional elements 132, wireless transmitter 140, and/or wireless receiver 142, and could also include data from said capsule 100, such as impedance data from impedance element 120, pressure data from pressure sensor 130, and/or pH data, visual data, etc., from one or more additional elements 132, as referenced herein. Console 200 can communicate directly with capsule 100, as referenced above, or indirectly through a network 400, such as shown in FIG. 4, such as by way of operation of a network element 230 configured to allow data/information to be transmitted between capsule 100 and console 200 through a network 400, such as the internet or an intranet.

An exemplary system 280 of the present disclosure, as shown in FIG. 2, therefore can comprise a capsule 100 and a console 200 and other items connected to console 200, such as an input device 250 and/or an output device 252.

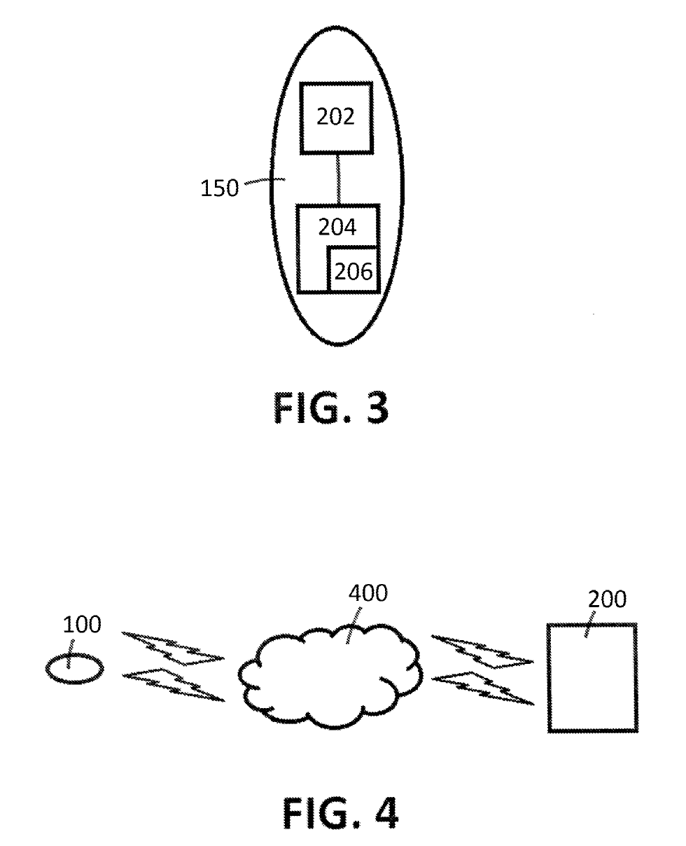

FIG. 3 shows elements of an exemplary computer element 150 referenced herein. As shown in FIG. 3, computer element 150 may comprise a processor 202 (such as a microprocessor) connected to or otherwise in communication with a storage medium 204, and software 206 (computer instructions) can be stored within storage medium 204 and accessed by processor 202 to operate various capsule 100 components, such as impedance element 120 (including, for example, electrodes 122, 124, 126, and/or 128), pressure sensor 130, one or more additional elements 132, wireless transmitter 140, and/or wireless receiver 142, as described herein.

While various embodiments of autoexpandable devices for obtaining impedance, pressure, and other measurements within the gastrointestinal tract and other organs and methods for using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A capsule, comprising:
an outer shell configured to dissolve or otherwise be digested within a stomach after ingestion;
an expandable material positioned within the outer shell, the expandable material configured for expansion within the stomach after the outer shell has dissolved; and
at least one element or sensor selected from the group consisting of an impedance element and a pressure sensor;
wherein the expandable material is surrounded by a mesh configured to retain the expandable material within said mesh.

2. The capsule of claim 1, wherein the at least one element or sensor comprises the impedance element or the pressure sensor.

3-7. (canceled)

8. The capsule of claim 1, wherein the at least one element or sensor comprises the impedance element and the pressure sensor, the impedance element comprising two detection electrodes positioned in between two excitation electrodes, wherein the impedance element is configured to obtain impedance data within a gastrointestinal tract, and wherein the pressure sensor is configured to obtain pressure data within a gastrointestinal tract.

9. The capsule of claim 1, wherein the at least one element or sensor is/are coupled to the expandable material.

10. The capsule of claim 1, further comprising at least one additional item, the at least one additional item selected from the group consisting of a camera configured to obtain visual data within a gastrointestinal tract, a pH sensor, a radiopaque marker, an electromagnetic marker, a mucosa-attached electromagnetic stimulator, a strain gauge having chemosensors, a wireless transmitter, a wireless receiver, a printed circuit configured to measure electrical/electromyography activity, and a gyroscope.

11-15. (canceled)

16. The capsule of claim 1, wherein the expandable material comprises a fungus.

17. (canceled)

18. The capsule of claim 1, wherein the mesh is configured to expand as the expandable material expands.

19. The capsule of claim 18, wherein the at least one element or sensor is/are coupled to the mesh.

20-21. (canceled)

22. The capsule of claim 1:
wherein the at least one element or sensor comprises the impedance element and the pressure sensor;
wherein the impedance element comprises two detection electrodes positioned in between two excitation electrodes;
wherein the impedance element is configured to obtain impedance data within a gastrointestinal tract;
wherein the pressure sensor is configured to obtain pressure data within a gastrointestinal tract;
wherein the expandable material comprises a fungus; and
wherein the mesh is configured to expand as the expandable material expands.

23-25. (canceled)

26. The capsule of claim 1, further comprising a substrate, wherein at least one of the at least one element or sensor is coupled to the substrate.

27-29. (canceled)

30. The capsule of claim 1, configured to deliver electrical current to stimulate motility of a colon to induce defecation.

31-33. (canceled)

34. The capsule of claim 1, further comprising a movement element coupled thereto and/or embedded therein, the movement element configured to facilitate movement of the capsule within the stomach.

35. The capsule of claim 1, further comprising an attachment element coupled thereto and/or embedded therein, the attachment element configured to facilitate engagement and ultimate disengagement of the capsule to a portion of a gastrointestinal tract.

36. A capsule, comprising:
an outer shell configured to dissolve or otherwise be digested within a stomach after ingestion;
an expandable material positioned within the outer shell, the expandable material configured for expansion within the stomach after the outer shell has dissolved;
a mesh;
an impedance element configured to obtain impedance data within a gastrointestinal tract; and
a pressure sensor configured to obtain pressure data within the gastrointestinal tract;
wherein at least one of the impedance element and the pressure sensor is/are coupled to a substrate; and
wherein the substrate is surrounded by the mesh.

37-39. (canceled)

40. A method of obtaining data, comprising:
operating a capsule within a digestive tract, the capsule comprising:
an outer shell configured to dissolve or otherwise be digested within a stomach after ingestion,
an expandable material positioned within the outer shell, the expandable material configured for expansion within the stomach after the outer shell has dissolved,
an impedance element, and
a pressure sensor;
wherein the step of operating is performed to obtain impedance data from the impedance element and pressure data from the pressure sensor.

41. (canceled)

42. The method of claim 40, further comprising the step of:
electrically stimulating a colon of the digestive tract using the capsule to induce defecation.

43. The capsule of claim 36, wherein the mesh surrounds the expandable material and is configured to expand as expandable material expands.

44. The capsule of claim 36, wherein the substrate is configured to remain within the capsule.

45. The capsule of claim 26, wherein the substrate is surrounded by the mesh.

46. The capsule of claim 26, wherein the mesh is configured to retain the substrate.
